(19) United States
(12) Patent Application Publication
(10) Pub. No.: US 2005/0115572 A1
(43) Pub. Date: Jun. 2, 2005

(54) Electrically activated alteration of body tissue stiffness for breathing disorders

(76) Inventors: Stephen N. Brooks, San Francisco, CA (US); Jed E. Black, Stanford, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/982,759

(22) Filed: Nov. 4, 2004

Related U.S. Application Data
(63) Continuation-in-part of application No. 10/679,935, filed on Oct. 6, 2003.
(60) Provisional application No. 60/517,164, filed on Nov. 5, 2003.
(60) Provisional application No. 60/415,995, filed on Oct. 4, 2002.

(51) Int. Cl. A62B 7/00; A61M 16/00

(52) U.S. Cl. 128/863

(57) ABSTRACT

Medical devices, systems, and methods mitigate a variety of disorders, including sleep-related breathing disorders. A stiffness, shape, and/or size of a reinforced tissue structure can be altered by applying a magnetic field and/or electrical field. The upper airway can be remodeled at night while maintaining physiological movement (such as swallowing, speaking, singing, and the like) when awake. Biasing of the tissue structures may also be employed.

### CROSS-REFERENCES TO RELATED APPLICATIONS

This application is a continuation-in-part of U.S. patent application Ser. No. 10/679,935, filed Oct. 6, 2003 (Atty. Docket No. 025625-000110US), and entitled “System and method for preventing Closure of Passageways;” which claims the benefit of U.S. provisional patent application Ser. No. 60/415,995, filed Oct. 4, 2002 (Atty. Docket No. 025625-000100US); and this application also claims the benefit of U.S. provisional patent application 60/517,164, filed on Nov. 5, 2003 (Atty. Docket No. 025625-000120US), and entitled “Method for Altering the Stiffness of Body Tissue or Organs;” each of which is herein incorporated by reference.

### BACKGROUND OF THE INVENTION

1. Field of the Invention

The invention is generally related to medical devices, systems, and methods, often reversibly and/or permanently altering the structural properties of tissues so as to change stiffness, shape, and/or size, particularly for tissues of the upper airway (as well as other tissue systems.)

Embodiments of the present invention generally relate to inhibition and/or prevention of abnormal breathing sounds (e.g., snoring); adverse consequences, illness or death in persons due to partial or complete blockage of the upper airway; or increased airflow resistance of the upper airway.

2. Description of the Related Art

A common and potentially serious disorder in humans involves involuntary closure of the airway during sleep. This disorder is known as “sleep-disordered breathing” or “obstructive sleep apnea” (OSA). In persons with OSA, there is involuntary closure or reduction in caliber of a portion of the airway that connects the atmosphere to the lungs. The upper portion of the airway (the “upper airway”) consists of two passageways, the nasal airway and the oral airway. These two passageways merge to become a single passageway. Portions of the upper airway just behind the tongue are known as the soft palate, the pharynx, the hypopharynx, etc.

In persons affected by OSA, closure, reduction in patency or increased airflow resistance of the upper airway occurs during sleep, due to a combination of physiological changes associated with sleep (including relaxation of muscles) and the anatomy of the upper airway (which is generally smaller or more crowded than in normal individuals). In persons prone to sleep apnea, a portion or portions of the muscular walls of the upper airway may become narrow or collapse, leading to reduction in airflow (“hypopnea”), cessation of airflow (“apnea”), increase in airflow turbulence or increased resistance to airflow within the airway. In the instance of collapse, the upper airway is blocked, breathing stops, air movement to the lungs ceases, and the oxygen level in the blood tends to decrease. As a response to this process (or to less severe manifestations, such as hypopneas or increased airway resistance), a brief arousal usually occurs in the brain. As a consequence of the brief arousal, the muscle tone in the walls of the upper airway returns to waking levels, and the airway abnormality is corrected—i.e. airway resistance and patency return to normal levels.

Generally, following each event, the patient returns to sleep, until another partial or complete upper airway collapse occurs and the process repeats itself. Depending on the severity in an individual case, the number of events may range from a few per hour of sleep to more than 100 events per hour of sleep. This process disrupts normal sleep. As a consequence, patients typically suffer from the effects of sleep deprivation. Such effects may include daytime drowsiness, tiredness or fatigue, difficulties with mental concentration or memory, mood changes, reductions in performance or increases in mistakes, and increased risk of accidents. Additionally, OSA is known to increase the risk of development of other medical problems.

Snoring is a mild form of sleep-disordered breathing in which increased airflow turbulence occurs. The snoring sounds result from tissue vibration within the nasal or oral airway. While snoring has been traditionally regarded as a social or cosmetic problem, recent studies suggest that snoring may be linked to the development of health problems, including high blood pressure.

Airway closure during sleep generally occurs at one or both of two levels in the upper airway: the soft palate and the hypopharynx (base of the tongue). At either level, the anterior tissue can collapse against the posterior pharyngeal wall, which makes up the rear wall of the throat. Additionally, the side (lateral) walls of the upper airway can collapse inward partially, or completely against each other. The lateral walls of the airway are susceptible to collapse in many patients with obstructive sleep apnea and other forms of sleep-related breathing disorders. In these cases, prevention of collapse of the airway only in the anterior-posterior dimension is insufficient to maintain normal airway patency. Even after extensive airway surgery for sleep apnea (which primarily addresses the anterior-posterior dimension of the airway), the patient may continue to have problems with breathing during sleep, due to lateral wall collapse or dysfunction.

Several types of treatment are available for obstructive sleep apnea and other sleep-related breathing disorders. The most common treatment consists of an air pressure delivery system that applies greater than atmospheric pressure to all walls of the upper airway to reduce the potential for full or partial collapse. Many people have difficulty using this device or prefer not to use it for various reasons. Also, surgical reconstruction of the airway or dental devices may be used. These treatments, however, often fail to treat the problem adequately.

Accordingly, a need exists in the art for an improved method and system for treating sleep apnea and other sleep-related breathing disorders. More generally, new devices, systems, and methods for altering the structural properties tissues would be beneficial, particularly where these techniques could be implemented without inhibiting the physiological functions performed by the tissues.

### BRIEF SUMMARY OF THE INVENTION

Novel medical devices, systems, and methods are provided which may find applications for mitigating a variety of disorders, including sleep-related breathing disorders. Some of these techniques allow structural properties of tissues to be selectively and/or intermittent modified, particularly by altering a stiffness, shape, and/or size of a reinforced tissue structure. The invention may take advantage of shape memory alloys or polymers, ferromagnetic polymers, ferrogels, electrically activated polymers, electro-rheostatic, piezoelectric, and/or magneto-rheostatic materials, and the like, with these materials often changing the structural characteristics of the reinforced tissue when a field (typically a magnetic field and/or electrical field) is applied. By allowing the structural stiffening of tissue systems of the upper airway to be modified at selected times, sleep-related breathing disorders can be mitigated while allowing physiological movement (such as swallowing, speaking, singing, and the like) at other times (such as during a portion of a sleep cycle or breathing cycle, and particularly when awake). Biasing of the tissue structures toward an open position may also be employed. Embodiments of the present invention are generally directed to a system for treating sleep-related breathing disorders. Materials of fixed stiffness may be attached to portions of the walls of the upper airway so as to maintain upper airway patency, and reinforcement of other anatomical structures which would benefit from added rigidity or stiffness (including but not limited to the penis and the heart) may also be provided.

In one embodiment, the system includes a first magnet attached to a left lateral pharyngeal wall, and a second magnet attached to a right lateral pharyngeal wall. The second magnet is positioned opposite the first magnet across an upper airway.

In another embodiment, the system includes a first magnetically susceptible material attached to a left lateral pharyngeal wall and a second magnetically susceptible material attached to a right lateral pharyngeal wall. The second magnetically susceptible material is positioned opposite the first magnetically susceptible material across an upper airway. The system further includes a first magnet disposed outside the body and lateral to the first magnetically susceptible material, and a second magnet disposed outside the body and lateral to the second magnetically susceptible material.

In yet another embodiment, the system includes a first magnet attached to a left lateral pharyngeal wall and a second magnet attached to a right lateral pharyngeal wall. The second magnet is positioned opposite the first magnet across an upper airway. The system further includes a third magnet disposed inside the upper airway directly across from the first magnet and a fourth magnet disposed inside the upper airway directly across from the second magnet.

In another aspect, the invention provides a method for inhibiting a sleep-related breathing disorder of a patient. The patient has an airway with an airway wall, and the method comprises attaching a material to the airway wall. The attached material is reversibly stiffened so that the stiffened attached material mitigates the sleep-related breathing disorder.

The attached material may be plastically deformable prior to and/or after stiffening. The attached material may have a liquid, gel, or pliable configuration and a stiffened configuration, with the attached material in the liquid, gel, or pliable configuration having sufficient flexibility to deform with an adjacent region of the airway during physiological movement. The attached material in the stiffened configuration may inhibit hypermobility or resonant movement of the adjacent region sufficiently to mitigate the sleep-related breathing disorder. Reversibly stiffening the attached material may change the attached material from the liquid, gel, or pliable configuration to the stiffened configuration. The method will often involve changing the material from the stiffened configuration to the liquid, gel, or pliable configuration, typically with the configuration of the material changing back and forth between the configurations repeatedly. The stiffened configuration may be used primarily or entirely while sleeping, and the stiffened configuration may be used throughout sleep or during only a portion of the sleep time (such as during portions of a sleep cycle or portions of a breathing cycle) so as to intermittently inhibit the breathing disorder while facilitating physiological movement.

The attached material may have a shape immediately prior to stiffening, and the stiffening may inhibit changes from the shape. The stiffening can, but need not impart a desired shape on the attached material so that the attached material does not necessarily impose a force against the airway wall after stiffening and prior to movement of the airway wall. In some embodiments, the material may comprise a magneto-rheostatic material ferromagnetic polymer, ferrogel, or the like, and the attached material may be stiffened by applying a magnetic field thereto. The attached material may optionally be biased with the magnetic field so as to open the airway, so that force may be applied by the attached material in some embodiments. In other embodiments, the material may comprise an electro-rheostatic material, electrically activated polymer, shape-memory polymer, or the like, and the attached material may be stiffened by applying an electrical field. Application of an electrical field may comprise applying an electrical current through the material using conductors coupling an electrical source to the material. A variety of alternative materials may be employed, including superelastic materials, shape memory alloys, piezoelectric materials, and the like, with combinations of these differing materials optionally being used in some embodiments.

The material may be attached by suturing the material to an upper airway wall, bonding the material to the upper airway wall, inserting the material into the upper airway wall, and/or the like. In many embodiments, the material will be inserted submucosally into the pharyngeal wall or other structure along the upper airway. The material may be inserted by penetrating a mucosa of the airway with a sharp distal tip extending from an insertion shaft. The material may be advanced distally to a target region using the insertion shaft and detached from the shaft so that the shaft can be withdrawn proximally from the patient. Material may be inserted through a plurality of mucosal penetration sites, with the attached material optionally defining a stiffening array. In some embodiments, the material may comprise a film such as a mesh or the like. The mucosa may be cut with an edge and a major surface of the film may be aligned along an adjacent surface of the airway.

In some embodiments, a stiffness of the attached material may be selected from among a plurality of alternative stiffnesses. The stiffening may change the material to the selected stiffness. The stiffness may be selected by varying the stiffness while monitoring the sleep-related breathing disorder so that sufficient stiffness is provided to inhibit the sleep-related breathing disorder without overly stiffening the airway, thereby titrating the stiffness.

Optionally, an energy supply can be implanted into the patient, with the attached material being stiffened by activating the energy supply (such as by completing a circuit between the energy supply and the attached material, an electromagnet, or the like). The energy supply may apply a magnetic field to the attached material, may apply an electrical field (and optionally an electrical current) to the attached material through a conductor, or the like. The energy supply may be implanted at least in part under a muscle of the neck, under skin of the chest or back, or the like, and may comprise a battery, a control circuit, and/or an electrical coupler configured for receiving electrical energy through skin.

In another aspect, the invention provides a system for inhibiting a sleep-related breathing disorder of a patient. The patient has an airway with an airway wall. The system comprises a material configured to be attached to an adjacent region of the airway wall. The material has a first configuration and a second configuration. The material in the first configuration provides the region with sufficient flexibility to deform during physiological movement when the material is attached to the airway wall. The attached material in the second configuration changes in stiffness, shape, or size to inhibit hypermobility or resonant movement of the adjacent region sufficiently to mitigate the sleep-related breathing disorder. The system also includes a source for generating a field. The field is capable of reversibly changing the material between the first configuration and the second configuration.

When the material comprises a ferromagnetic polymer, a ferrogel, or a magneto-rheostatic material, the source will typically comprise a magnetic field source. The field may be sufficient to induce biasing of the attached material so as to open the airway. The source may comprise an implantable magnetic field source for removably transmitting the magnetic field to the attached material from inside the patient body. In other embodiments, the source may comprise an external magnetic source, often accompanied by a support for removably mounting the source outside the patient body, such as a collar to be worn around the neck at night or the like. The material may again comprise electrically activated polymers, an electro-rheostatic material (typically stiffened by applying an electrical field and/or current), a superelastic material, and a piezoelastic material, as well as a magneto-rheostatic material.

The system may include a suture for suturing the material to the upper airway wall, adhesive for bonding the material to the upper airway wall, a probe for inserting the material into the upper airway wall, or the like. The probe may comprise a shaft supporting a sharp distal tip for penetrating a mucosa of the airway passage, typically under visual guidance (though other imaging modalities may also be employed, including endoscopes, ultrasound, optical coherence tomography, fluoroscopy, magnetic resonance imaging, and the like). The material may be advancable with the shaft into the airway wall for submucosal release and implantation. In other embodiments, the material may comprise a film, with the system optionally including an edge for cutting the mucosa, the film often being alignable with a major surface of the film extending within the airway wall along an adjacent surface region of the airway.

The source may comprise a variable source and may generate a variable field. A stiffness of the material in the second configuration may vary in response to the field so as to provide a plurality of alternative stiffness configurations. The source may have an input for varying the stiffness while monitoring the sleep-related breathing disorder. The source will often comprise an energy supply implantable into the patient. Activation of the energy supply may stiffen the material when the material is attached to the airway. The energy supply may apply a magnetic field, electrical current, and/or electrical field to the attached material. The energy supply may be coupled to the attached material by a conductor, and at least a portion of the energy supply may be implanted under a muscle of the neck, under skin of the chest or back, and the like. The energy supply may comprise a battery and/or an electrical coupler configured for receiving electrical energy through skin.

The material may comprise any of a variety of configurations, including a polymer, a plate, a bar, a sphere, and a plurality of pieces. The material may optionally comprise a mesh or other film. In some embodiments, the material may comprise at least one of a contained colloid, contained suspension, contained gel, or contained liquid. The colloid, suspension, gel, or liquid may comprise an electro-rheostatic or magneto-rheostatic material, and a biocompatible polymer, such as a polyester or PTFE, may encase the material.

In another aspect, the invention provides a method for treating a sleep-related breathing disorder of a patient. The patient has pharyngeal walls, and the method comprises attaching a magneto-rheostatic material to the pharyngeal walls. A magnetic field is applied to the attached material so that, during nighttime, stiffening of the attached material inhibits the sleep-related breathing disorder of the patient. The magnetic field is removed from the attached material during daytime.

In yet another aspect, the invention provides a system comprising a material configured to be attached to a tissue of a patient. The material comprises a magneto-rheostatic material having a first configuration and a second configuration. The material in the first configuration has sufficient flexibility to deform with physiological movement when the material is attached to the tissue. The attached material in the second configuration has a stiffness that is greater than in the first configuration. A source generates a magnetic field, and the field is capable of reversibly changing the material between the first configuration and a second configuration when the material is attached to the tissue.

The material may optionally comprise a contained colloid, suspension, gel, or liquid, often with a biocompatible polymer encasing the material. In other embodiments, the magneto-rheostatic material may comprise a polymer that remains solid in both the first and second configurations.

In another aspect, the invention provides a method for inhibiting a sleep-related breathing disorder of a patient. The patient has an airway with an airway wall, and the method comprises attaching a material to the airway wall. The breathing of the patient is monitored, and the attached material is reversibly stiffened, reversibly re-sized, or reversibly re-shaped in response to the monitoring so that the attached material mitigates the sleep-related breathing disorder. Optionally, a control circuit having a sensor transmits a signal to a field source so as to effect the change in the material.

In yet another aspect, the invention provides a system for inhibiting a sleep-related breathing disorder of a patient. The patient has an airway with an airway wall, and the system comprises a sensor for monitoring the patient. A material is configured to be attached to an adjacent region of the airway wall, the material having a first configuration and second configuration. The material in the first configuration allows physiological movement of the adjacent region of the airway wall when the material is attached. The attached material in the second configuration has a stiffness, shape, or size inhibiting hypermobility or resonant movement of the adjacent region sufficiently to mitigate the sleep-related breathing disorder. A source is often coupled to the sensor, the source generating a field capable of reversibly changing the material between the first configuration in response to the monitoring.

In another aspect, the invention provides a method for inhibiting a sleep-related breathing disorder of a patient. The patient has an airway with an airway wall, and the method comprises attaching a material to the airway wall. The attached material is reversibly stiffened by altering an electrical field applied to the material so that the stiffened attached material mitigates the sleep related breathing disorder.

In yet another aspect, the invention provides a system for inhibiting a sleep-related breathing disorder of a patient. The patient has an airway with an airway wall, and the system comprises an electro-rheostatic material configured to be attached to the airway wall. The material has a first configuration and a second configuration, the material in the first configuration having sufficient flexibility to deform with an adjacent region of the airway during physiological movement when the material is attached to the airway wall. The attached material in the second configuration has a stiffness inhibiting hypermobility or resonant movement of the adjacent region sufficiently to mitigate the sleep-related breathing disorder. A source generates an electrical field, the field capable of reversibly changing the material between the first configuration and a second configuration.

In another method aspect, the invention provides a method for inhibiting a sleep-related breathing disorder of a patient. The patient has an airway with an airway wall, and the method comprises attaching a material to the airway wall. A breathing characteristic of the patient is monitored, and an electrical field is reversibly applied to the attached material in response to the monitoring so that the attached material changes configuration and mitigates the sleep related breathing disorder.

In a final aspect, the invention provides a system for inhibiting a sleep-related breathing disorder of a patient. The patient has an airway with an airway wall, and the system comprises a material configured to be attached to the airway wall, the material having a first configuration and second configuration. The material in the first configuration allows deformation of an adjacent region of the airway during physiological movement when the material is attached to the airway wall. The attached material in the second configuration inhibits hypermobility or resonant movement of the adjacent region sufficiently to mitigate the sleep-related breathing disorder. A sensor monitors a breathing characteristic of the patient, and a source is coupled to the sensor so as to generate an electrical field in response to the monitoring. The field is capable of reversibly changing the material between the first configuration and a second configuration.

### BRIEF DESCRIPTION OF THE DRAWINGS

The following detailed description makes reference to the accompanying drawings, which are now briefly described.

FIGS. 1A, 1B and 3-5 illustrate a series of coronal views of an upper airway, each having a system for treating sleep-related breathing disorders in accordance with one embodiment of the invention.

FIG. 2 illustrates a sagittal view of the upper airway having a system for treating sleep-related breathing disorders in accordance with one embodiment of the invention.

FIG. 6 schematically depicts placement of materials of fixed or variable stiffness attached to the walls of the upper airway, along with field source devices for transmitting a field toward the variable stiffness materials.

FIGS. 7A and 7B are cross-sectional views showing tissues disposed along an upper airway for a patient having normal sleep-related breathing and a patient having an abnormal airway associated with snoring, sleep apnea, or other sleep-related breathing disorders.

FIG. 8 schematically illustrates variable stiffness materials attached to tissues along an upper airway by implantation of the materials, as shown in a lateral cross-sectional diagram.

FIG. 9 schematically illustrates a coronal view of an upper airway passage having variable stiffness materials implanted therein, along with external field source devices transmitting a field to the variable stiffness materials from outside the patient body.

FIG. 10 schematically illustrates a method for attaching a stiffening material to an airway wall under direct visualization, and also illustrates a probe for penetrating a mucosa of the airway wall and introducing a stiffening material.

FIG. 11 is a detailed view schematically illustrating insertion of a reinforcing or stiffening structure within a wall of the upper airway, and also illustrates a stiffening array formed by a plurality of discrete stiffening members.

FIG. 12 is a schematic coronal view of an upper airway illustrating attached materials for stiffening an airway wall, along with structures of electrical field and/or magnetic field sources so as to controllably and reversibly allow the attached materials to be stiffened and returned to their flexible configuration from inside the patient body and/or outside the patient body.

FIG. 13 schematically illustrates an exemplary material to be attached to an upper airway wall, with the exemplary material including an electro-rheostatic or magneto-rheostatic liquid, gel, colloid, or suspension contained within an elongate polymer casing.

FIG. 14 schematically illustrates a variable stiffness mesh, with the fibers of the mesh comprising electro-rheostatic or magneto-rheostatic materials encased in a polymer.

FIG. 15 schematically illustrates implanting a variable stiffness mesh into an upper airway passage.

FIGS. 16A and 16B illustrate changing a configuration of an electro-rheostatic or magneto-rheostatic material from a first configuration to a second configuration, in which the second configuration has a greater stiffness than the first configuration.

While the invention is described herein by way of example for several embodiments and illustrative drawings, those skilled in the art will recognize that the invention is not limited to the embodiments or drawings described. It should be understood, that the drawings and detailed description thereto are not intended to limit the invention to the particular form disclosed, but on the contrary, the intention is to cover all modifications, equivalents and alternatives falling within the spirit and scope of the present invention as defined by the appended claims. The headings used herein are for organizational purposes only and are not meant to be used to limit the scope of the description or the claims. As used throughout this application, the word “may” is used in a permissive sense (i.e., meaning having the potential to), rather than the mandatory sense (i.e., meaning must). Similarly, the words “include”, “including”, and “includes” mean including, but not limited to.

### DETAILED DESCRIPTION OF THE INVENTION

As used herein, “attaching” a material to a tissue structure (such as an airway wall or the like) encompasses inserting, implanting, and/or embedding the material into the tissue structure, as well as affixing the tissue structure to an exposed surface of the tissue structure or the like.

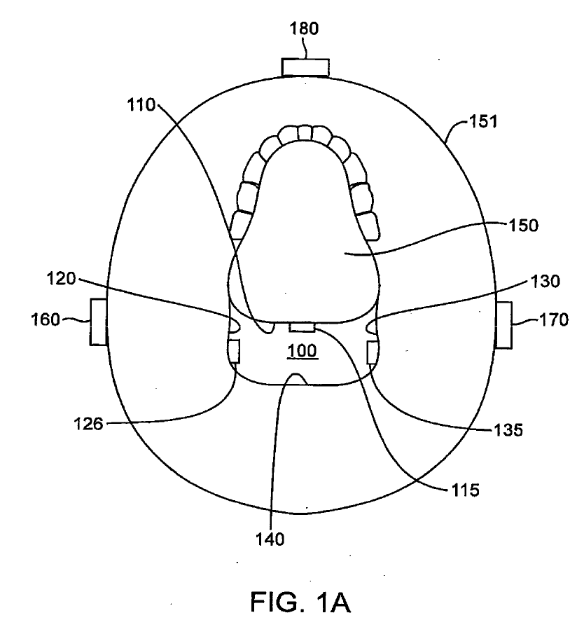

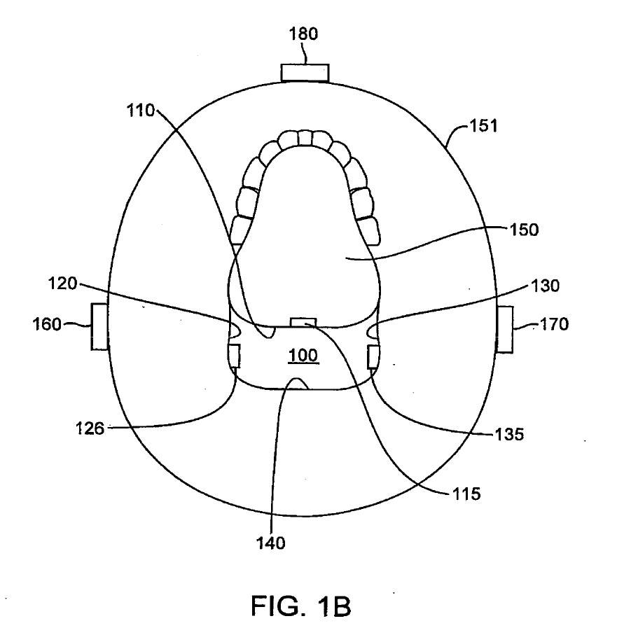

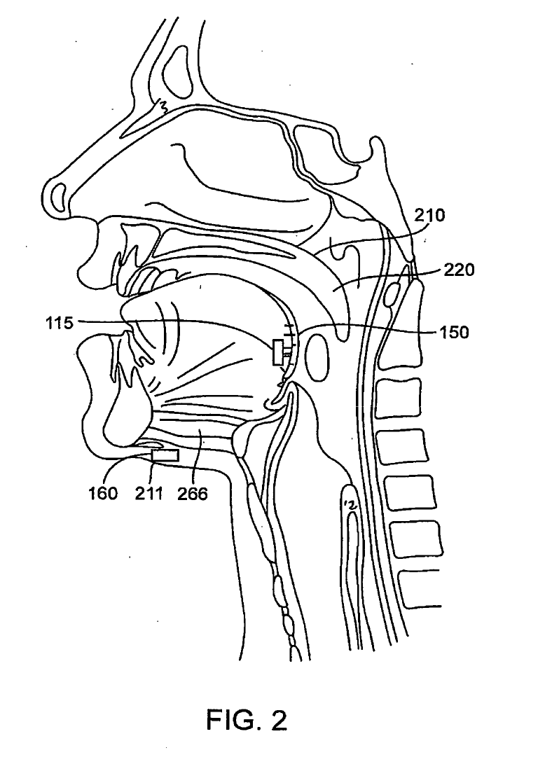

FIG. 1A illustrates a coronal view of an upper airway 100 having a system for treating sleep apnea (and other sleep-related breathing disorders, e.g., snoring) in accordance with one embodiment of the invention. The upper airway 100 is generally defined by the anterior pharyngeal wall 110, two lateral pharyngeal walls 120, 130 and the posterior pharyngeal wall 140. The lateral pharyngeal walls 120, 130 generally include lateral pharyngeal tissue extending superiorly to the velopharynx and inferiorly to the epiglottis. The posterior pharyngeal wall 140 generally includes posterior pharyngeal tissue extending superiorly to the velopharynx and inferiorly to the epiglottis. The anterior pharyngeal wall 110 generally includes a base portion of the tongue 150, the soft palate 210 and the uvula 220 (shown in FIG. 2). Magnetically susceptible material 115 is attached to the anterior pharyngeal wall 110, magnetically susceptible material 125 is attached to the lateral pharyngeal wall 120, and magnetically susceptible material 135 is attached to the lateral pharyngeal wall 130. In one embodiment, magnetically susceptible materials 115, 125, 135 are attached to the respective pharyngeal walls by surgical sutures or bonding material, such as surgical glue. Other means for attaching the magnetically susceptible materials to the pharyngeal walls are also contemplated by embodiments of the invention described herein. In another embodiment, the magnetically susceptible materials 115, 125, 135 may be implanted inside, or embedded beneath the surface of, the respective pharyngeal walls, as shown in FIG. 1B. In yet another embodiment, the magnetically susceptible materials 115, 125, 135 may be coated on the surfaces of the respective pharyngeal walls.

The magnetically susceptible materials 115, 125, 135 may be materials, which are not magnets, but are susceptible to magnetic fields, such as ferromagnetic materials. As such, magnetically susceptible materials 115, 125, 135 would not interact with each other in the absence of a magnetic field, such as, during daytime, as opposed to permanent magnets that would potentially interact with each other at all times, which may be inappropriate or even deleterious (e.g., during speaking or swallowing) to a person's health. Magnetically susceptible materials 115, 125, 135 may be in the form of plates, discs, spheres, bars, multiple small pieces, mesh and the like. In an alternate embodiment, the magnetically susceptible materials 115, 125, 135 may be replaced with magnets, such as permanent magnets with magnetic fields of fixed strength or variable magnets (e.g., electromagnets) with magnetic fields of variable strength (including zero if not activated).

Magnet 160 is positioned outside the body and lateral to magnetically susceptible material 125, while magnet 170 is positioned outside the body and lateral to magnetically susceptible material 135, and magnet 180 is positioned outside the body and anterior to magnetically susceptible material 115. Magnets 160, 170, 180 may be attached or placed adjacent to the outer skin 151 of a patient with means, such as a neckband or a chin strap. In one embodiment, magnets 160, 170, 180 may be implanted beneath the outer skin surface, such as, beneath the front skin 211 of the cheek 266 for magnet 160, as shown in FIG. 2.

Magnet 160 is configured to attract magnetically susceptible material 125 toward magnet 160 so that movement of the lateral pharyngeal wall 120 toward closure of the upper airway 100 may be opposed. Magnet 170 is configured to attract magnetically susceptible material 135 toward magnet 170 so that movement of the lateral pharyngeal wall 130 toward closure of the upper airway 100 may be opposed. Magnet 180 is configured to attract magnetically susceptible material 115 toward magnet 180 so that movement of the anterior pharyngeal wall 110 toward closure of the upper airway 100 may be opposed. In this manner, the cross sectional dimensions (e.g., the length or width) of the upper airway 100 may be increased or prevented from decreasing, thereby allowing patency of the upper airway 100 to be maintained.

Force fields between magnet 160 and magnetically susceptible material 125 and between magnet 170 and magnetically susceptible material 135 act to keep the soft tissue of the lateral pharyngeal walls 120, 130 from collapsing. Force fields between magnet 180 and magnetically susceptible material 115 act to keep the soft tissue of the anterior pharyngeal wall 110 from collapsing toward the posterior pharyngeal wall 140.

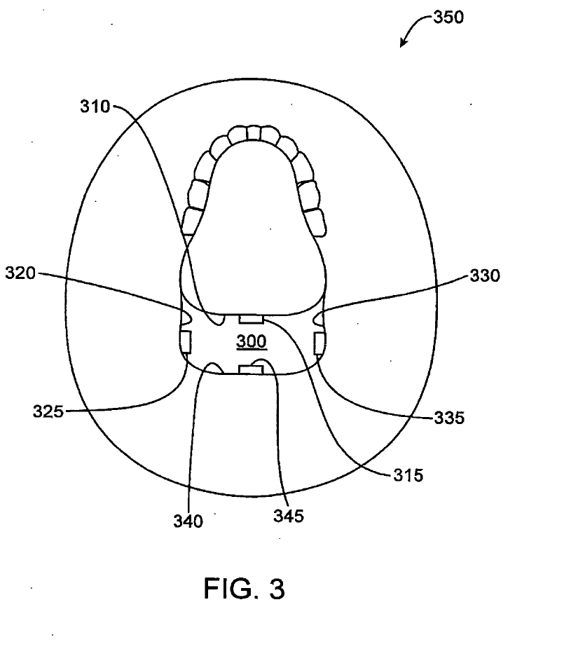

FIG. 3 illustrates a coronal view of an upper airway 300 having a system 350 for treating sleep apnea (and other sleep-related breathing disorders, e.g., snoring) in accordance with another embodiment of the invention. The system 350 includes magnet 315 attached to an anterior pharyngeal wall 310, magnet 325 attached to lateral pharyngeal wall 320, magnet 335 attached to lateral pharyngeal wall 330, and magnet 345 attached to posterior pharyngeal wall 340. In one embodiment, magnets 315, 325, 335, 345 are attached to the respective pharyngeal walls by surgical sutures or bonding material, such as surgical glue. Other means for attaching the magnets to the pharyngeal walls are also contemplated by embodiments of the invention described herein. In another embodiment, magnets 315, 325, 335, 345 may be implanted inside (e.g., embedded beneath the surface of) the respective pharyngeal walls. In yet another embodiment, magnets 315, 325, 335, 345 may be coated on surfaces of the respective pharyngeal walls.

Magnets 315, 325, 335, 345 may be permanent magnets with magnetic fields of fixed strength or variable magnets, such as electromagnets, with magnetic fields of variable strength (including zero if not activated).

Magnets 315, 325, 335, 345 are oriented such that the same magnetic poles of the magnets 315, 325, 335, 345 face each other, e.g., north poles facing other north poles. In operation, magnets 315, 325, 335, 345 are configured to repel each other, thereby opposing closure of the upper airway 300 without the use of external magnets.

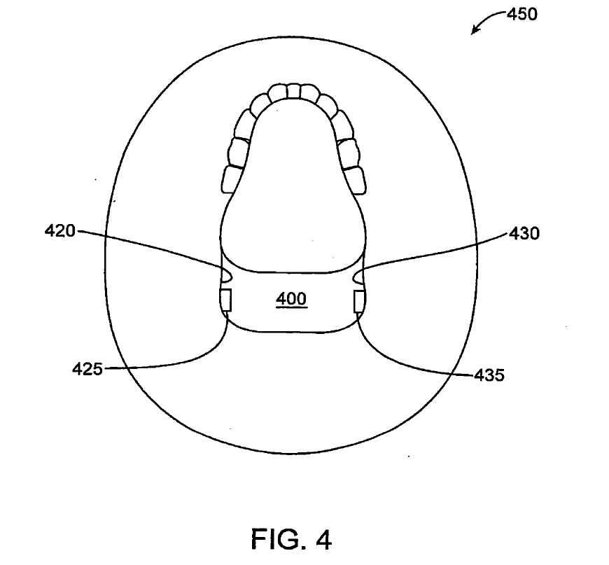

FIG. 4 illustrates a coronal view of an upper airway 400 having a system 450 for treating sleep apnea (and other sleep-related breathing disorders, e.g., snoring) in accordance with yet another embodiment of the invention. The system 450 includes magnet 425 attached to lateral pharyngeal wall 420 and magnet 435 attached to lateral pharyngeal wall 430. In one embodiment, magnets 425, 435 are attached to the respective lateral pharyngeal walls by surgical sutures or bonding material, such as surgical glue. Other means for attaching the magnets to the lateral pharyngeal walls are also contemplated by embodiments of the invention described herein. In another embodiment, magnets 425, 435 may be implanted inside (e.g., embedded beneath the surface of) the respective lateral pharyngeal walls. In yet another embodiment, magnets 425, 435 may be coated on surfaces of the respective lateral pharyngeal walls.

Magnets 425, 435 may be permanent magnets with magnetic fields of fixed strength or variable magnets, such as electromagnets, with magnetic fields of variable strength (including zero if not activated). Magnets 425, 435 are oriented such that the same magnetic poles of the magnets 425, 435 face each other, e.g., north pole facing other north pole. In operation, magnets 425, 435 are configured to repel each other, thereby opposing closure of the upper airway 400 without the use of external magnets.

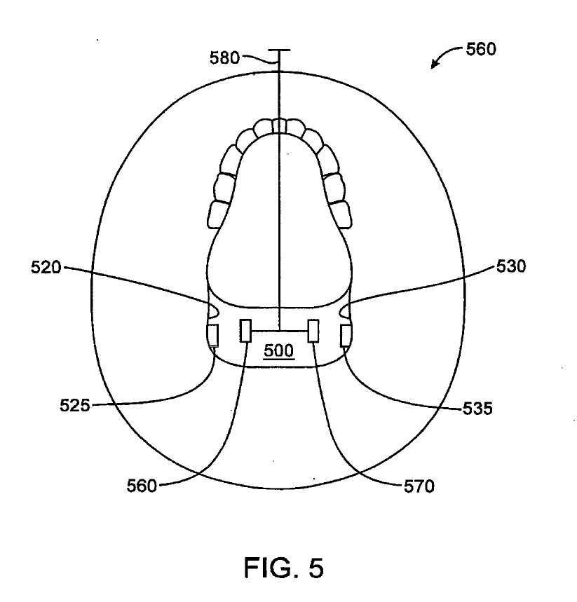

FIG. 5 illustrates a system 550 for treating sleep apnea (and other sleep-related breathing disorders, e.g., snoring) disposed inside an upper airway 500 in accordance with still another embodiment of the invention. The system 550 includes magnet 525 attached to lateral pharyngeal wall 520 and magnet 535 attached to lateral pharyngeal wall 530. In one embodiment, magnets 525, 535 may be attached to the lateral pharyngeal walls 530, 535 by surgical sutures or bonding material, such as surgical glue. Other means for attaching the magnets to the pharyngeal walls are also contemplated by embodiments of the invention described herein. In another embodiment, magnets 525, 535 may be implanted inside the lateral pharyngeal walls 530, 535. In yet another embodiment, magnets 525, 535 may be coated on surfaces of the lateral pharyngeal walls 530, 535. Magnets 525, 535 may be permanent magnets with magnetic fields of fixed strength or variable magnets, such as electromagnets, with magnetic fields of variable strength (including zero if not activated).

The system 550 further includes magnets 560 and 570 disposed inside the upper airway 500. Magnet 560 is disposed across from magnet 525, while magnet 570 is disposed across from magnet 535. The magnetic poles of magnets 560, 570 are oriented such that magnets 560, 570 repel magnets 525, 535, respectively, thereby opposing closure of the upper airway 500 without the use of external magnets. Magnets 560, 570 may be attached to or held in place by a removable apparatus 580, such as a mouthpiece.

Each magnet or magnetically susceptible material described herein may comprise more than one magnet or magnetically susceptible material. Although embodiments of the invention have been described with reference to two or four magnetically susceptible materials or magnets, embodiments of the invention also contemplate other combinations or numbers of magnets and magnetically susceptible materials. Although embodiments of the invention have been described with reference to treating sleep-related breathing disorders, such as sleep apnea or snoring, embodiments of the invention also contemplate other applications where passageway or airway patency is required. For example, the magnets or magnetically susceptible materials may be inserted or attached through a body aperture, such as the vagina, the rectum, the urinary passage and the like.

A method is also described for altering the stiffness or rigidity of tissues or organs of the body, either temporarily or permanently. Such a methodology is beneficial for maintaining patency of the upper airway, by using materials that increase the stiffness of the airway. This process would be primarily useful in the treatment of sleep-related breathing disorders, in which airway patency tends to decrease or airway resistance tends to increase during sleep, resulting in breathing impairment and various negative impacts on health, physical and cognitive functions and quality of life. The changes in the airway during sleep result, in part, because of relaxation of muscle tissue comprising the walls of the upper airway.

Said method would also be useful in the alleviation of snoring, by stabilizing and reducing vibration in tissues of the upper airway.

In one embodiment of the invention, materials of fixed stiffness are attached to portions of the walls of the upper airway, by sutures, bonding material or temporary or permanent coating. Said substances might have various configurations, including, but not limited to, plates, bars, small spheres, multiple small pieces, mesh or contained colloid, suspension, gel or liquid.

In another embodiment of the invention, materials of fixed stiffness are implanted within portions of the walls of the upper airway. Said substances might have various configurations, including, but not limited to, plates, bars, small spheres, multiple small pieces, mesh or contained colloid, suspension, gel or liquid.

In still another embodiment of the invention, materials of variable stiffness, shape, and/or size are attached to portions of the walls of the upper airway, by sutures, bonding material, or temporary or permanent coating. The stiffness (and/or size) of such materials can be increased by application of electric current (in the case of so-called “piezoelectric” or “electro-rheostatic” materials) or magnetic field(s) (in the case of so-called “magneto-rheostatic” materials). Said substances might have various configurations, including, but not limited to, plates, bars, small spheres, multiple small pieces, mesh or contained colloid, suspension, gel or liquid. Electric current(s) or magnetic field(s) may originate from devices such as batteries and/or electromagnets placed within or in close proximity to the materials or from devices placed external to the body.

In still another embodiment of the invention, materials of variable stiffness, shape, and/or size are implanted within portions of the walls of the upper airway. The stiffness (and/or size) of such materials can be increased by application of electric current (in the case of so-called “piezoelectric” or “electro-rheostatic” materials) or magnetic field(s) (in the case of so-called “magneto-rheostatic” materials). Said substances might have various configurations, including, but not limited to, plates, bars, small spheres, multiple small pieces, mesh or contained colloid, suspension, gel or liquid. Electric current(s) or magnetic field(s) may originate from devices such as batteries, fixed magnets, and/or electromagnets placed within or in close proximity to the materials or from devices placed external to the body.

Increasing the stiffness of the walls of the upper airway during sleep is intended to maintain upper airway patency during sleep, treat sleep-related breathing disorders (including snoring) and prevent the adverse consequences that are known to result from such disorders.

Said method might also find application in the manipulation of other anatomical structures, which require or would benefit from added rigidity or stiffness, including but not limited to the penis and the heart.

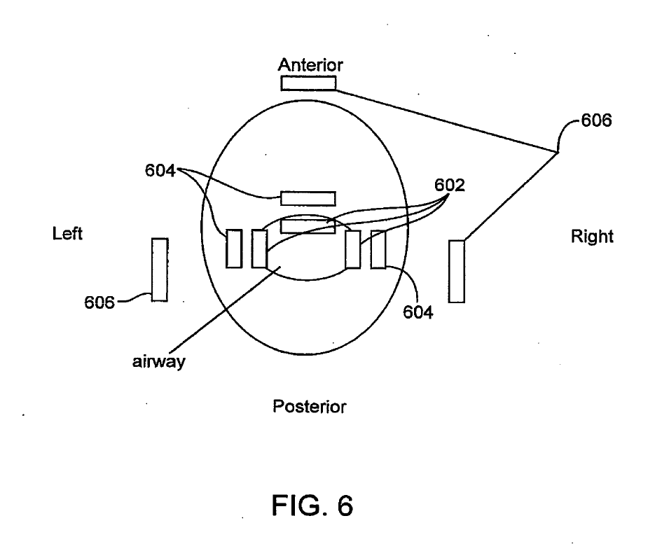

FIG. 6 depicts placement of materials of fixed or variable stiffness 602 attached to the walls of the upper airway or 604 implanted in the walls of the upper airway. Materials of variable stiffness may be acted upon by electric current(s) or magnetic field(s), originating from external devices 606.

FIGS. 7A and 7B schematically illustrate some of the tissue structures disposed along the upper airway, and show typical differences between those structures in a normal upper airway (FIG. 7A) and an abnormal upper airway (FIG. 7B) of a patient suffering from a sleep-related breathing disorder. The abnormal tissues defining the upper airway wall often intrude into the airway, with many disorders being related to obesity. As the tissues protrude into the airway, the speed of airflow during breathing and the like increases within the narrowed passage. Per Bernoulli's equation, the pressure on the passage walls decreases with increasing flow velocities, potentially pulling the walls further into the passage. As described above, forces may optionally be applied to the airway walls so as to increase the size of the passage. However, most patients with sleep-related breathing disorders do not suffer from interruption of airflow during the day, in part because tensing of the muscle tissues may stiffen the passage sufficiently to inhibit hypermobility and/or resonant movement. Relaxation of the muscles at night decreases their stiffness, allowing them to intrude into the airway and/or vibrate.

So as to avoid interfering with normal physiological movement of the tissues along the upper airway, it may be advantageous to avoid permanently stiffening tissues sufficiently to inhibit breathing disorders. The variable stiffness reinforcing structures, systems, and methods described herein may allow stiffening to be effected in a controlled manner, for example, with stiffening of the tissues by the reinforcing material being greater at nighttime than during the day, optionally being greater at selected portions of the nighttime (such as in response to snoring sounds, movement of the airway passage tissues within a predetermined frequency range, or the like). Along with (or instead of) stiffening of the walls of the upper airway, changes in size and/or shape of a reinforcing material may also be employed to mitigate the sleep-related breathing disorder. In some embodiments, stiffening, re-sizing, and/or reshaping of the tissue reinforcing materials may be implemented in response to signals generated by a sensor. Hence, stiffening, re-sizing, and/or reshaping may optionally occur only at times of acute breathing disruption, during selected portions of a sleep cycle, or during selected portions of a respiration cycle.

Along with selecting times for enhancing stiffness, changing size, or altering shape, the variable reinforcement materials described herein may allow varying of the structural properties of the attached material throughout a range of stiffness, size, and/or shape settings, to any of a plurality of alternative discrete stiffnesses, sizes, or shapes, or the like. For example, by varying an intensity of a magnetic field applied to a magneto-rheostatic material, the stiffness of the material may be controllably varied. Optionally, the magneto-rheostatic material may comprise a plurality of magneto-rheostatic components which are stiffened at differing magnetic field thresholds. Still further alternatives may be provided, including both a magneto-rheostatic material and electro-rheostatic material, with one level of stiffness being provided by application of a magnetic field, and a second, greater stiffness being provided by application of both magnetic and electrical fields. Still further alternative modes for controllably varying stiffness may be implemented by varying an electrical field strength, an electrical current, or the like.

As the tissues along the upper airway move with swallowing and other physiological movement, and as patients may swallow while asleep, it may be advantageous to limit stiffening of the attached materials so as to provide an effective amount of stiffening, without overly inhibiting physiological movement. Toward that end, after sufficient variable stiffness material has been attached at the appropriate locations along an upper airway passage, the activating field (often magnetic and/or electrical) that is applied to the attached material may be varied, with the stiffness (for example) being gradually increased until the sleep-related breathing disorder is sufficiently mitigated. This effectively titrates the stiffening of the airway passage, thereby providing a therapy which can be tailored to a specific patient. In some embodiments, selected attached materials may be stiffened while others are not, or to a greater extent than others. Hence, still further refinements and tailoring of the therapy may be provided by the controllable variable stiffness materials described herein. Titrating and tailoring of changes in size and/or shape of reinforcement materials may similarly be effected.

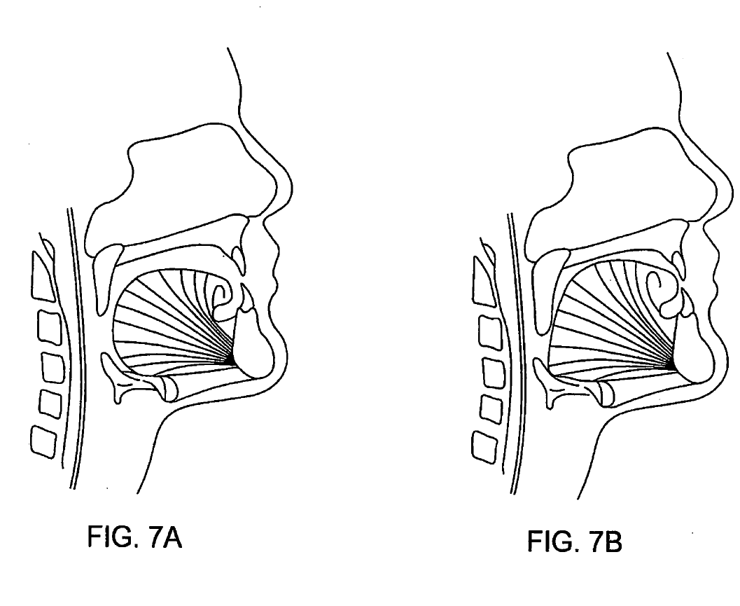

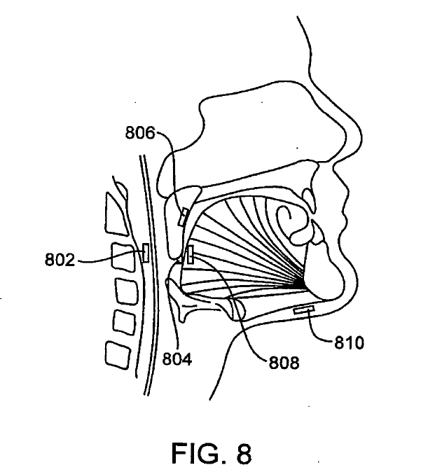

FIG. 8 schematically illustrates some of the locations for attaching variable support materials along an upper airway passage, with the locations here being shown in a schematic sagittal view. More specifically, variable stiffening, variable size, and/or variable shape material 802 is attached to the posterior pharyngeal wall and material 804 is attached to the lateral pharyngeal wall. Material 806 is attached to the uvula, while material 808 is attached to the posterior portion of the tongue or epiglottis. Still further locations are possible, including along the lower jaw 810.

The different locations for attaching variable reinforcement material may be particularly well suited for different forms or orientations of materials, and may be used to produce different airway-altering effects. For example, material 902 may optionally comprise a piezoelectric or other variable size material, and may elongate laterally when an electrical field is applied so as to inhibit lateral pharyngeal wall collapse. Material 804 may extend in an anterior/posterior orientation, and may comprise an electro-rheostatic or magneto-rheostatic material so as to stiffen the lateral walls when the material is exposed to an electrical or magnetic field. Alternatively, material 804 may comprise a shape memory polymer or a shape memory alloy extending along an anterior/posterior and/or superior/inferior length, and may change in shape, optionally in concavity or convexity, in response to an electrical field so as to increase an open cross-section of the airway wall. Advantageously, electrical activation of shape memory polymers may be associate with little or no heating of adjacent tissues, and may also alter a stiffness of the material, with or without changing a shape of the attached material. Still further alternatives are possible, including forming material 804 using variable size materials configured to be positioned and oriented so as to inhibit posterior movement of the tongue when a field is applied, such as by pushing tongue and/or tongue-supporting tissues in an anterior direction.

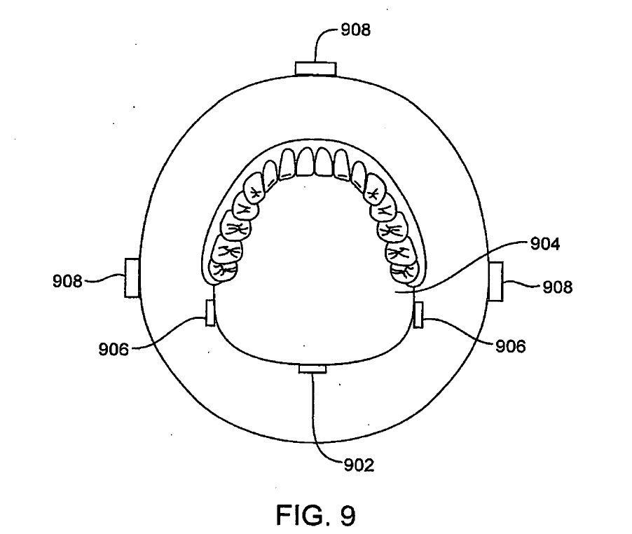

Referring now to FIG. 9, a coronal view illustrates variable stiffness, shape, and/or size material attached to an anterior pharyngeal wall 902, a posterior pharyngeal wall, epiglottis, or posterior of the tongue 904 and the lateral pharyngeal walls 906. While embodiments are generally described below as using variable stiffness materials, the size and/or shape of the material may instead be controllably varied, with or without also varying a stiffness of the material. The attached materials may also provide variable stiffness at least in part due to the tissue response to the attached materials. For example, tissue ingrowth and/or scar tissue formation my help stiffen the reinforced tissue. Alternatively, piezoelectric materials may be attached and an electrical current applied so as to elongate the piezoelectric material. Although the piezoelectric material may not itself stiffen when elongated, the adjacent tissue may be distended so that the tissue/material combination is effectively stiffened when an electrical current is applied. Still further alternatives include variable shape materials such as shape memory polymers and the like, which may stiffen as well as change shape.

External field sources 908 are distributed about (or slightly above) the neck and apply sufficient fields to stiffen the attached materials 902, 904, 906. The sources may comprise permanent magnets, electromagnets, or the like, and may be supported by a collar worn around the neck. Variable stiffness attached materials 902, 904, 906 may be attached to the airway passage walls by bonding (such as using any of a wide variety of surgical adhesives, including cyanoacrylate-derived materials, fibrin-based adhesives, or the like), suture or other mechanical fasteners (such as surgical staples, or the like) by temporary or permanent coating of the airway wall with the material, or by implanting the materials into the walls of the airway passage.

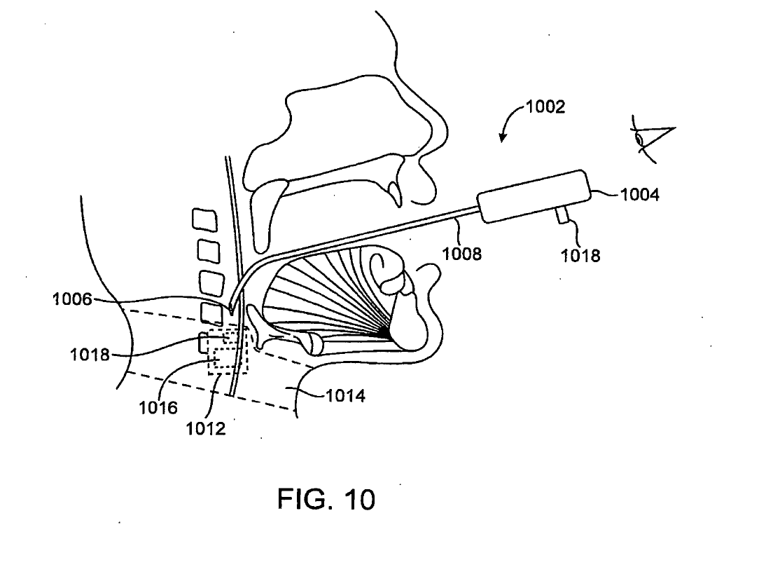

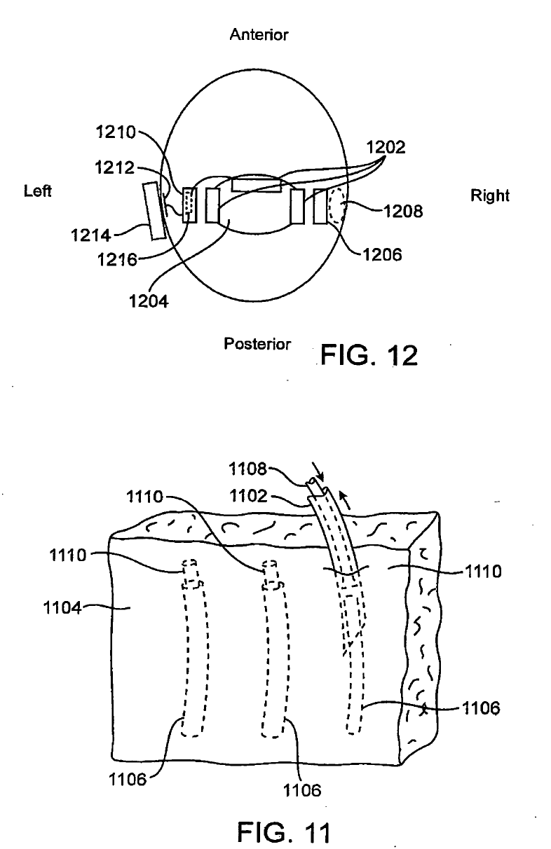

Referring now to FIGS. 10 and 11, a method and probe for inserting variable stiffness and other reinforcing materials into the tissues along an upper airway passage are schematically illustrated. Probe 1002 generally has a proximal handle 1004 coupled to a sharpened distal tip 1006 by a shaft 1008. A physician advances tip 1006 into the posterior pharyngeal wall while directly viewing the penetration site. As can be understood with reference to FIGS. 10 and 11, a distal portion 1102 of probe 1002 is advanced into and through a mucosa 1104 and into an underlying layer of the airway passage wall. Once the tip of the probe has been advanced so that a variable stiffness material 1106 within the probe is disposed in a target region of the pharyngeal wall tissue, the variable stiffness material can be implanted by withdrawing the probe proximally while holding the material in place using an inner shaft 1108 of the probe. Handle 1004 of probe 1002 will often have an actuator 1018 for moving inner shaft 1108 relative to outer shaft 1102. A wide variety of alternative probe structures may be used to implant the variable stiffness material into the airway wall, including structures similar to those used for brachytherapy.

As is also illustrated in FIG. 11, a plurality of discrete bodies of variable stiffness (or other support characteristic) material 1106 may be implanted through an associated plurality of mucosal penetration sites 1110. The material forms an array for stiffening the adjacent airway passage wall. In some embodiments, elongate bodies of variable stiffness material may be aligned in laterally offset arrays as shown. Other embodiments may make use of bodies that are axially offset, that are angularly offset, that cross, or the like.

Also seen in outline in FIG. 10 is a field generation device 1012 for applying a field to a variable stiffness material so as to change the material from a first, liquid, gel, or pliable configuration to a second, more rigid configuration. Field source 1012 is, at least in part, supported by a collar 1014 worn around a neck of the patient. Source 1012 may include a battery 1016 and a field transmission surface 1018. The field transmission surface may comprise a fixed magnet surface, and/or a surface be coupled to an electromagnet (in the case of magnetically susceptible variable stiffness materials). Source device 1012 may optionally be used to both stiffen a variable stiffness material and bias the material so as to move the tissues of the airway passage to an open position, as described above.

FIG. 12 schematically illustrates additional aspects of a system for inhibiting sleep-related breathing disorders, and particularly of sources for generating fields so as to reversibly change a material attached to an airway passage tissue from a first, pliable or even liquid configuration to a second, stiffer configuration. The attached material 1202 is again illustrated schematically as being disposed along anterior and lateral walls of airway 1204. An implanted field source 1206 has been implanted beneath a muscle 1208 adjacent to (but separated from) a portion of the attached material 1202. Source 1206 may be disposed below the muscle, and the implantation site may be accessed from an external approach. The field transmitted from source 1206 to the adjacent attached material 1202 may be transmitted through the intervening tissue therebetween, or an electrical or magnetic conductor may extend between the two structures. Such a conductor is shown extending from field source 1210 to anterior attached material 1202. Source 1210 also has another conductor extending to a through-skin electrical coupler 1212 which is adapted to provide electrical power for the field source.

Optionally, energy for the field source may be provided directly from connector 1212 by (for example) wearing a collar having a corresponding energizing circuit or connector 1214 during the night. When energizing circuit 1214 is placed outside the skin adjacent coupler 1212, energy can be delivered safely through the skin using, for example, corresponding external and internal coils. In other embodiments, the external energy source may be used to charge a battery implanted with the field source. Regardless, electrical and/or magnetic fields may be applied without having to repeatedly penetrate the tissue. Suitable structures for charging or energizing the source have been developed for charging cardiac pacemakers, implantable insulin and other drug delivery pumps, artificial heart and/or heart-assisting devices, and the like.

Implanted field source 1210 includes a control circuit 1216 coupling energy source 1214 or battery to the field transmission surface and/or conductor. Control circuit 1216 may have a memory or other tangible media embodying machine-readable programming code for implementing any one or more of the methods described herein. Control circuit 1216 may comprise a digital and/or analog circuit, and may have a reprogrammable memory so as to allow modifications to tissue stiffening treatment regime. Communication with implanted field source 1210 may be implemented by a wireless transmitter and/or receiver of control circuit 1216, by signals transmitted using coupler 1212, or the like, and the control circuit may also include sensors for detecting snoring and/or apnea, timing circuits, variable field strength controllers, and other components explicitly or implicitly described herein. The control circuit (including the sensor) and tangible media may partly or fully included in implanted field source 1210, partly or fully included in external energy source 1214, and/or in another structure in communication with one or both of these structures in any of a wide variety of alternative data processing architectures.

Control circuit 1216 may apply a field so as to alter a stiffness, size, or shape of attached material 1202 in response to signals from the sensor of the circuit. The sensor may comprise any of a wide variety of structures, and may monitor breathing by detecting one or more of a number of different patient parameters. Exemplary sensors may detect changes in sound (for example, the sensor comprising a microphone), changes in vibration (with the sensor comprising an accelerometer or the like), turbulence of the airflow, flow resistance, airway diameter, body position, respitory events (such as apneas or hypobneas), oxygen saturation (optionally using pulse oximetry), respiration effort, brain wave activity, electrophysiological heart signals, or the like. Control circuit can alter the size, shape, or stiffness of the attached material in response to one or more of these monitored characteristics meeting or exceeding a threshold value, and/or at cycle intervals (such as periodically during selected portions of the respiration cycle, the sleep cycle, or the like).

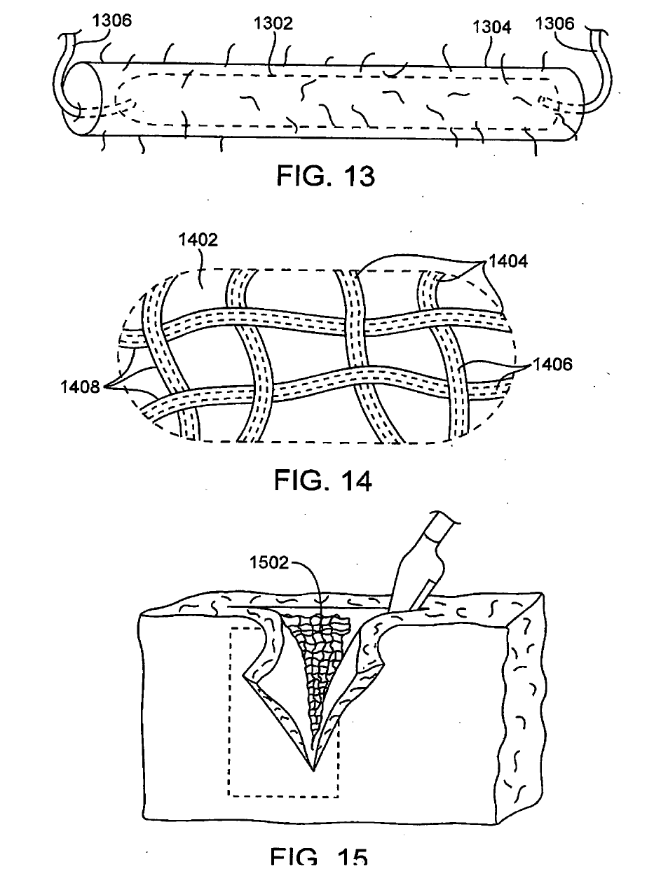

Referring now to FIG. 13, an exemplary structure of a stiffening material is shown in more detail. In this embodiment, a variable stiffness (or other property) material comprises a colloid, suspension, liquid, or gel 1302 contained within an outer polymer casing 1304. Variable stiffness material 1302 will often comprise a magneto-rheostatic and/or electro-rheostatic material. Such materials are sometimes included within the general category of “smart materials” and have physical properties which can be significantly and controllably altered. Electro-rheostatic and magneto-rheostatic materials are often fluids, and can experience a dramatic change in their viscosity, often changing from a thick fluid (similar to motor oil) to a solid or near-solid substance within times of about one millisecond or less when exposed to a magnetic or electric field. The effect is often reversible just as quickly when the magnetic field is removed.

The most common form of magneto-rheostatic fluid comprises minute iron particles suspended in oil. Magneto-rheostatic fluids have been developed for use in car shocks, damping machine vibrations, prosthetic limbs, and the like. Magneto-rheostatic materials suitable for use as variable stiffness materials in the present invention may be commercially available from Lord Corp., located in Cary, N.C., with exemplary products being sold under the trademark Rheonetic™ systems and materials. Electro-rheostatic materials have been developed for use in clutches and valves, as well as for structures intended to reduce noise and vibration. Electro-rheostatic materials may be as simple as milk chocolate or cornstarch and oil. Along with Lord Corp., SRI of Menlo Park, Calif.; mnemoScience GmbH of Aachen Germany, Mide Corp., Morgan Electro Ceramics of Bedford Ohio, and others are developing and/or commercializing polymers which change shape, size, or stiffness when electrical or magnetic fields are applied, as well as piezoelectric materials and/or shape memory alloys which may find applications in embodiments described herein.

Ideally, the variable stiffness material 1302 will be biocompatible so as to limit any damage to the patient should the material leak from casing 1304. In some embodiments, the material may comprise a solid prior to stiffening, so that the material need not necessarily be encased. Nonetheless, it will often be advantageous to provide a casing over the variable stiffness material so as to insure an appropriate tissue response to the implanted or attached structure. Casing 1304 may comprise a polyester, a PTFE, or the like, and may have external fibers or surface pores so as to promote tissue ingrowth to help affix the material to the adjacent tissues. As described above, conductors 1306 may extend between the variable stiffness material and the field source so as to apply an appropriate electrical field, electrical current, magnetic field, or the like.

Referring now to FIG. 14, variable stiffness material 1402 here takes the form of a mesh. The individual fibers 1404 of mesh 1402 each comprise a contained polymer, colloid, suspension, liquid, or gel 1406 disposed within a casing 1408. Mesh 1402 may be affixed to a surface of the upper airway or implanted within an upper airway wall as schematically illustrated in FIG. 15. As with many of the variable stiffness materials described herein, the mesh structure 1502 may be highly flexible or pliable prior to application of an electrical or magnetic field thereto. Upon application of an appropriate field, the mesh or other variable stiffness materials may stiffen in whatever configuration or shape the materials define at that time, particularly when stiffening is effected by changing phase of the material within an outer case from a liquid to a solid. The materials may, at least in part, deform plastically prior to stiffening and/or when in the stiffened configuration.

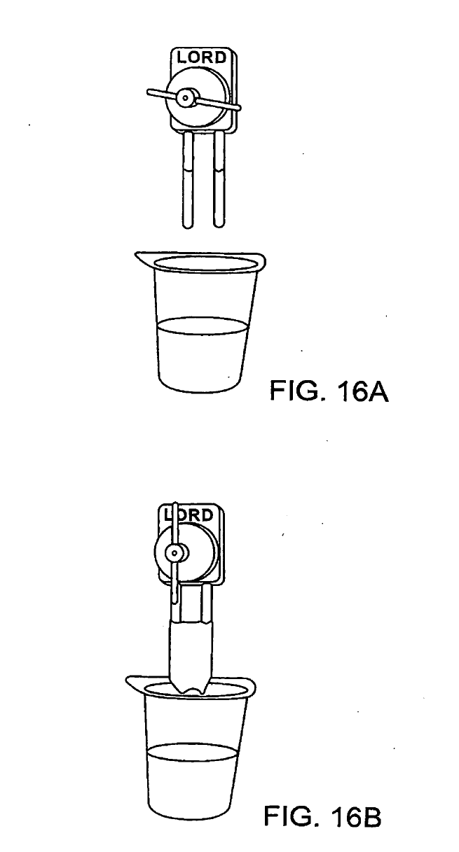

Referring now to FIGS. 16A and 16B, solidifying a viscous electro-rheostatic or magneto-rheostatic material can be seen, with the material forming a solid upon application of the appropriate field. In some smart materials, stiffening may be effected by removing a field, or the like.

While the foregoing is directed to embodiments of the present invention, other and further embodiments of the invention may be devised without departing from the basic scope thereof, and the scope thereof is determined by the claims that follow.

1. A method for inhibiting a sleep-related breathing disorder of a patient, the patient having an airway with an airway wall, the method comprising:
attaching a material to the airway wall; and
reversibly stiffening the attached material by altering an electrical field or current applied to the material so that the stiffened attached material mitigates the sleep related breathing disorder.

2. The method of claim 1, wherein the attached material is plastically deformable.

3. The method of claim 2, wherein the attached material has a pliable configuration and a stiffened configuration, the attached material in the pliable configuration having sufficient flexibility to deform with an adjacent region of the airway during physiological movement, the attached material in the stiffened configuration inhibiting hypermobility or resonant movement of the adjacent region sufficiently to mitigate the sleep-related breathing disorder, wherein reversibly stiffening the attached material changes the attached material from the pliable configuration to the stiffened configuration.

4. The method of claim 3, further comprising changing the attached material from the stiffened configuration to the pliable configuration by removing the electrical field or current.

5. The method of claim 1, wherein the attached material has a shape immediately prior to stiffening, and wherein the stiffening inhibits changes from the shape.

6. The method of claim 5, wherein the stiffening does not impart a desired shape on the attached material so that the attached material does not impose a force against the airway wall after stiffening and prior to movement of the airway wall.

7. The method of claim 1, wherein the material comprises an electro-rheostatic material and wherein the attached material is stiffened by applying the electrical field.

8. The method of claim 1, wherein the material comprises at least one of a superelastic material, electrically activated polymer, and a piezoelectric material.

9. The method of claim 1, wherein the material is attached by at least one of suturing the material to the upper airway wall or tongue, bonding the material to the upper airway wall or tongue, and inserting the material into the upper airway wall or tongue.

10. The method of claim 9, wherein the material is attached by inserting the material submucosally into the pharyngeal wall.

11. The method of claim 10, wherein the material in inserted submucosally by penetrating a mucosa with a sharp distal tip extending from an insertion shaft, advancing the material to a target region using the insertion shaft, detaching the material from the insertion shaft; and withdrawing the insertion shaft proximally from the patient.

12. The method of claim 10, wherein the materially is inserted through a plurality of mucosal penetration sites, the attached material defining a stiffening array.

13. The method of claim 10, wherein the material comprises a film, and further comprising cutting the mucosa with an edge and aligning a major surface of the film along an adjacent surface of the airway.

14. The method of claim 1, further comprising selecting a stiffness from among a plurality of alternative stiffnesses of the material, the stiffening changing the material to the selected stiffness.

15. The method of claim 14, wherein the stiffness is selected by varying the stiffness while monitoring the sleep-related breathing disorder.

16. The method of claim 1, further comprising implanting an energy supply into the patient, wherein the attached material is stiffened by activating the energy supply.

17. The method of claim 16, wherein the activation energy supply is coupled to the attached material by a conductor.

18. The method of claim 16, wherein the energy supply is implanted at least in part under a muscle of the neck, or subcutaneously along an upper chest wall front or back.

19. The method of claim 16, wherein the energy supply comprises at least one of a battery and an electrical coupler configured for receiving electrical energy through skin, and further comprising a control circuit electrically coupling the energy supply to the attached material, the control circuit having a sensor, wherein the sensor monitors a sleep characteristic of the patient and transmits a signal to effect changes in the stiffness.

20. A system for inhibiting a sleep-related breathing disorder of a patient, the patient having an airway with an airway wall, the system comprising:
an electro-rheostatic material configured to be attached to the airway wall, the material having a first configuration and second configuration, the material in the first configuration having sufficient flexibility to deform with an adjacent region of the airway during physiological movement when the material is attached to the airway wall, the attached material in the second configuration having a stiffness inhibiting hypermobility or resonant movement of the adjacent region sufficiently to mitigate the sleep-related breathing disorder; and
a source generating an electrical field, the field capable of reversibly changing the material between the first configuration and a second configuration.

21. The system of claim 20, wherein the material is plastically deformable.

22. The system of claim 20, wherein the attached material in the first configuration is pliable.

23. The system of claim 20, wherein the attached material has a shape immediately prior to changing from the first configuration to the second configuration, and wherein the change in configuration inhibits changes from the shape.

24. The system of claim 23, wherein the change in configuration does not impart a desired shape on the attached material so that the attached material does not impose a force against the airway wall after stiffening and prior to movement of the airway wall.

25. The system of claim 20, further comprising at least one of:
suture for suturing the material to the upper airway wall,
adhesive for bonding the material to the upper airway wall, and
a probe for inserting the material into the upper airway wall.

26. The system of claim 25, wherein the probe comprises a shaft supporting a sharp distal tip for penetrating a mucosa of the airway passage under visual guidance, the material advancable with the shaft and releasable submucosally.

27. The system of claim 26, wherein the materially comprises a plurality of separate bodies insertable through a plurality of mucosal penetration sites, so that the attached material defines a stiffening array.

28. The system of claim 20, wherein the material comprises a film.

29. The system of claim 20, wherein the source comprises a variable source and generates a variable field, a stiffness of the material in the second configuration varying in response to the field so as to provide a plurality of alternative stiffness configurations.

30. The system of claim 29, wherein the source has an input for varying the stiffness while monitoring the sleep-related breathing disorder.

31. The system of claim 20, wherein the source comprises an energy supply implantable into the patient, wherein activation of the energy supply stiffens the material when the material is attached to the airway passage.

32. The system of claim 31, further comprising an implantable conductor for coupling the energy supply to the attached material.

33. The system of claim 31, wherein the energy supply is implantable at least in part under a muscle of the neck.

34. The system of claim 31, wherein the energy supply comprises at least one of a battery and an electrical coupler configured for receiving electrical energy through skin, and further comprising a control circuit electrically coupling the energy supply to the attached material, the control circuit having a sensor, wherein the sensor monitors a sleep characteristic of the patient and transmits a signal to effect changes in the stiffness.

35. The system of claim 20, wherein the material comprises at least one of a polymer, a plate, a bar, a sphere, and a plurality of pieces.

36. The system of claim 20, wherein the material comprises a mesh.

37. The system of claim 20, wherein the material comprises at least one of a contained polymer, colloid, gel, or liquid.

38. The system of claim 37, wherein the contained colloid, gel, or liquid comprises at least one of a shape memory polymer, an electrically activated polymer, and an electro-rheostatic material, and further comprising a biocompatible polymer encasing the material.

39. A method for inhibiting a sleep-related breathing disorder of a patient, the patient having an airway with an airway wall, the method comprising:
attaching a material to the airway wall;
monitoring a breathing characteristic of the patient; and
reversibly applying an electrical field to the attached material in response to the monitoring so that the attached material changes configuration and mitigates the sleep related breathing disorder.

40. A system for inhibiting a sleep-related breathing disorder of a patient, the patient having an airway with an airway wall, the system comprising:
a material configured to be attached to the airway wall, the material having a first configuration and second configuration, the material in the first configuration allowing deformation of an adjacent region of the airway during physiological movement when the material is attached to the airway wall, the attached material in the second configuration inhibiting hypermobility or resonant movement of the adjacent region sufficiently to mitigate the sleep-related breathing disorder;
a sensor for monitoring a breathing characteristic of the patient; and
a source coupled to the sensor so as to generate an electrical field in response to the monitoring, the field capable of reversibly changing the material between the first configuration and a second configuration.
